(19) United States
(12) Patent Application Publication
(10) Pub. No.: US 2026/0021256 A1
(43) Pub. Date: Jan. 22, 2026

(54) MEDICINE INJECTION AND DISEASE MANAGEMENT SYSTEMS, DEVICES AND METHODS

(71) Applicant: Bigfoot Biomedical, Inc., Milpitas, CA (US)

(72) Inventors: Per John SJOLUND, Los Altos, CA (US); Lane DESBOROUGH, Thousand Oaks, CA (US); Bryan MAZLISH, Palo Alto, CA (US); Andrew BOCHENKO, Redwood City, CA (US); Ross NAYLOR, Fullerton, CA (US)

(21) Appl. No.: 19/246,557

(22) Filed: Jun. 23, 2025

Related U.S. Application Data
(63) Continuation of application No. 17/806,901, filed on Jun. 14, 2022, now Pat. No. 12,350,479.
Application No. 17/806,901 is a continuation of application No. 16/566,544, filed on Sep. 10, 2019, now Pat. No. 11,806,514.
Application No. 16/566,544 is a continuation of application No. 15/717,805, filed on Sep. 27, 2017, now Pat. No. 10,426,896.
(60) Provisional application No. 62/400,366, filed on Sep. 27, 2016.

(51) Int. Cl. A61M 5/32; A61M 5/172; G06F 3/04817; G06F 3/0482; G06F 3/04847; G06F 3/04883; G16H 10/60; G16H 20/10; G16H 20/17; G16H 40/63; G16H 50/20

(52) U.S. Cl. CPC A61M 5/3202; A61M 5/1723; G06F 3/04817; G06F 3/0482; G06F 3/04847; G16H 20/10; G16H 20/17; G16H 50/20; A61M 2205/3303; A61M 2205/3306; A61M 2205/3561; A61M 2205/50; A61M 2205/505; A61M 2205/52; A61M 2230/201; G06F 3/04883; G16H 10/60; G16H 40/63

(57) ABSTRACT

One or more embodiments of the present disclosure may include an insulin delivery system that includes an insulin delivery device, a user interface that includes multiple user-selectable icons or buttons each representing different meal characteristics, memory to store one or more user-specific dosage parameter, and a processor in communication with the memory and adapted to receive blood glucose data. The processor may also be adapted to determine initial meal characteristics associated with each of the user-selectable icons or buttons based on at least one of the user-specific dosage parameters. The processor may also be adapted to update the meal characteristics associated with each of the user-selectable icons or buttons based upon the blood glucose data.

### CROSS-REFERENCE TO RELATED APPLICATIONS

This application is a continuation of U.S. patent application Ser. No. 17/806,901, filed Jun. 14, 2022, continuation of U.S. patent application Ser. No. 16/566,544, filed Sep. 10, 2019, now U.S. Pat. No. 11,806,514, issued Nov. 7, 2023, which is a continuation of U.S. patent application Ser. No. 15/717,805, filed Sep. 27, 2017, now U.S. Pat. No. 10,426,896, issued Oct. 1, 2019, which claims the benefit of U.S. Patent Provisional Application No. 62/400,366, filed Sep. 27, 2016, the disclosure of each of which is hereby incorporated herein in its entirety by this reference.

### TECHNICAL FIELD

The present disclosure relates to medicine injection and disease management systems, devices, and methods, particularly those related to the management of diabetes and/or the delivery of insulin. In some embodiments, systems, methods, and devices provided herein can personalize user-selectable meal sizes for a user to enter meal data for purposes of obtaining recommendations regarding one or more medication doses. In some cases, one or more buttons (e.g., user-selectable icons, physical press buttons, etc.) can be personalized to describe an amount or range of amounts of insulin to be delivered and/or carbohydrates to be consumed.

### BACKGROUND

Diabetes mellitus is a chronic metabolic disorder caused by an inability of a person's pancreas to produce sufficient amounts of the hormone, insulin, such that the person's metabolism is unable to provide for the proper absorption of sugar and starch. This failure leads to hyperglycemia, i.e., the presence of an excessive amount of analyte, such as glucose, within the blood plasma. Persistent hyperglycemia has been associated with a variety of serious symptoms and life threatening long-term complications such as dehydration, ketoacidosis, diabetic coma, cardiovascular diseases, chronic renal failure, retinal damage and nerve damages with the risk of amputation of extremities. Self-monitoring of blood glucose and the self-administration of insulin is the typical method for treating diabetes. The “correct” insulin dosage is a function of the level of glucose in the blood. Insufficient insulin dosages can result in hyperglycemia, and excessive insulin dosages can result in hypoglycemia, which can result in clumsiness, trouble talking, confusion, loss of consciousness, seizures, or death. Accordingly, people with diabetes (PWDs) face a considerable cognitive burden in determining an appropriate dosage of insulin.

In order to assist with this self-treatment, many diabetes-related devices (e.g., blood glucose meters, insulin pumps, etc.) are equipped with insulin bolus calculators that have the user input a number of carbohydrates consumed (or about to be consumed) and the bolus calculator outputs a recommended size for the insulin bolus dosage. Although bolus calculators remove some of the calculations that need to be made by the user in determining an appropriate insulin bolus dosage, bolus calculators still burden the user with the mental task of determining the number of carbohydrates in their meal and often require manual entry of data. Accordingly, there is a need for methods, systems, and devices that further reduce the cognitive burden on the user while improving the accuracy of a recommended insulin bolus dosage.

### BRIEF SUMMARY

Systems, devices and methods provided herein can be equipped to simplify calculation of a recommended insulin dosage by simplifying a meal announcement process and/or simplifying the collection of an estimated glucose value (EGV). A meal announcement process can be simplified by providing a user interface that includes one or more meal announcement buttons, which can be user-selectable icons on a touchscreen, physical press buttons, jog dials, voice activation commands, etc. In some cases, meal announcement buttons can be located on part of an insulin delivery device or on an accessory for an insulin delivery device. In some cases, the insulin delivery device can be an insulin pen and a dose capture pen cap including the meal announcement buttons. In some cases, an insulin delivery device or an accessory therefor can be in wireless communication with a remote user interface device (e.g., a mobile application on a smartphone) and the meal announcement buttons can be on the remote user interface device (either as physical press buttons or as user-selectable icons on a touch screen). The one or more meal announcement buttons can be personalized based on user's use of the system. Such personalization may be based on a user interacting with various buttons or features (e.g., the user selecting various meal sizes for boluses), by providing boluses and measuring the effect for the boluses over time, or any other use of devices or systems in accordance with the present disclosure.

One or more embodiments of the present disclosure may include an insulin delivery system that includes an insulin delivery device, a user interface that includes multiple user-selectable icons or buttons each representing different meal characteristics, memory to store one or more user-specific dosage parameter, and a processor in communication with the memory and adapted to receive blood glucose data. The processor may also be adapted to determine initial meal characteristics associated with each of the user-selectable icons or buttons based on at least one of the user-specific dosage parameters. The processor may also be adapted to update the meal characteristics associated with each of the user-selectable icons or buttons based upon the blood glucose data.

In accordance with one or more devices, systems, or methods of the present disclosure, a device or system may include a blood glucose monitor or sensor.

In accordance with one or more devices, systems, or methods of the present disclosure, the systems or devices may include a flash glucose monitor that includes a flash near field communication circuit, and a system near field communication circuit in communication with the processor. In these and other embodiments, the processor may be adapted to receive the blood glucose data via near field communications (NFC) when the system near field communication circuit and the flash near field communication circuit are brought within an NFC communication distance.

In accordance with one or more devices, systems, or methods of the present disclosure, the systems or devices may include a continuous glucose monitor, and the processor may be adapted to receive wireless communications from the continuous glucose monitor at predetermined time intervals.

In accordance with one or more devices, systems, or methods of the present disclosure, the systems or devices may include a continuous glucose monitor, and the processor may be adapted to receive wireless communications from the continuous glucose monitor at predetermined time intervals.

In accordance with one or more devices, systems, or methods of the present disclosure, the user-selectable icons or buttons may each initially represent an amount of carbohydrates in 5 gram or 10 gram increments.

In accordance with one or more devices, systems, or methods of the present disclosure, the amount of carbohydrates initially represented by each of the plurality of icons may be determined based on an insulin Sensitivity Factor (ISF), a Carb Ratio (CR), a body weight, an age, a total daily basal (TDB) rate, a daily dosage of Long-Acting Insulin, a weight averaged total daily dosage (TDD) of insulin and/or a combination thereof of a person with diabetes (PWD).

In accordance with one or more devices, systems, or methods of the present disclosure, the processor may be further configured to determine an insulin delivery amount based on an amount of carbohydrates associated with a selected one of the user-selectable icons or buttons and/or the blood glucose data.

In accordance with one or more devices, systems, or methods of the present disclosure, the user-selectable icons or buttons may each represent a number of units of insulin that are needed to compensate for each meal, rounded to the nearest 0.5 units.

In accordance with one or more devices, systems, or methods of the present disclosure, the updating of the meal characteristics associated with each of the plurality of user-selectable icons or buttons may be determined from postprandial blood glucose data after the user has selected a given user-selectable icon or button.

In accordance with one or more devices, systems, or methods of the present disclosure, the systems or devices may include a flash glucose monitor that includes a flash near field communication circuit, and the systems or devices may further include one or more system near field communication circuits in communication with the processor. In these and other cases, the processor may be adapted to receive the postprandial blood glucose data via near field communications (NFC) when the one or more system near field communication circuits and the flash near field communication circuit are brought within an NFC communication distance. Additionally, the processor may be adapted to send a prompt to the user to retrieve the postprandial blood glucose data by bringing one of the one or more system near field communication circuits into close proximity to the flash glucose monitor at a predetermined time after insulin is delivered or one of the user-selectable icons or buttons has been selected by the user.

In accordance with one or more devices, systems, or methods of the present disclosure, the user interface may be adapted to display a bolus recommendation based on the blood glucose data and a selection of one of the user-selectable icons or buttons.

In accordance with one or more devices, systems, or methods of the present disclosure, the processor may determine the bolus recommendation based on factors selected from the number of carbohydrates divided by the PWD's carbohydrate-to-insulin ratio, a difference between the current blood glucose level and a target blood glucose level divided by the PWD's insulin sensitivity factor, a reading from a blood glucose meter (BGM), data from a continuous glucose monitor (CGM), blood glucose trend data, Insulin on Board (IOB) data, Carbohydrates on Board (COB) data, whether the PWD is or plans to exercise, whether the PWD is sick, whether the PWD is pregnant, whether the PWD is experiencing menses, and whether the PWD has consumed certain medications.

In accordance with one or more devices, systems, or methods of the present disclosure, the processor may be further adapted to receive dosage data from the insulin delivery device, and the update the meal characteristics associated with each of the user-selectable icons or buttons may be based upon postprandial blood glucose data after the user has selected that user-selectable icon or button, the dosage data, or a combination thereof.

In accordance with one or more devices, systems, or methods of the present disclosure, the insulin delivery device may include an insulin pen, and the user interface may be part of the insulin pen, part of a pen accessory adapted to reversibly connect to an insulin pen, or part of a mobile application for a smartphone in wireless communication with an insulin pen or an accessory therefore. In these and other embodiments, the devices or systems may be adapted to detect amounts of insulin remaining in or delivered by one or more insulin pens.

In accordance with one or more devices, systems, or methods of the present disclosure, the pen accessory may be adapted to reversibly connect to an insulin pen, where the pen accessory may include a pen cap that is adapted to detect amounts of insulin remaining in an insulin pen during placement or removal from the insulin pen or when secured to the insulin pen.

In accordance with one or more devices, systems, or methods of the present disclosure, the user interface may be located on the mobile application for a smartphone, where the smartphone further includes the processor, and the insulin pen or an accessory therefor is adapted to detect insulin amount or delivery data and wirelessly communicate the insulin amount or delivery data to the processor.

One or more embodiments of the present disclosure may include a cap for an insulin pen that includes one or more sensors adapted to detect the position of a plunger within an insulin pen, and a user interface that includes one or more user-selectable icons or buttons adapted to announce a meal or an intent to have a meal.

In accordance with one or more devices, systems, or methods of the present disclosure, the cap may include a processor and memory, where the processor may be adapted to determine a time and dosage for an insulin delivery based on data from the one or more sensors and store that information in the memory.

In accordance with one or more devices, systems, or methods of the present disclosure, the user interface may include at least 2, and no more than 6, user-selectable icons or buttons adapted to announce a meal or an intent to have a meal, each representing different meal characteristics stored for each button in the memory.

In accordance with one or more devices, systems, or methods of the present disclosure, the user interface may include a display adapted to display a recommended dosage based at least in part on a selection of the one or more user-selectable icons.

In accordance with one or more devices, systems, or methods of the present disclosure, the cap may include a wireless communication device adapted to communicate with a blood glucose monitor or sensor, where the display may be adapted to display a current blood glucose level, an indication of a current rate of change, a recommended correction bolus dosage based on glucose data, or a combination thereof.

In accordance with one or more devices, systems, or methods of the present disclosure, a wireless communication device of a cap may include an NFC circuit.

In accordance with one or more devices, systems, or methods of the present disclosure, the devices or systems may include an annunciator adapted to prompt the user to obtain blood glucose data from the blood glucose monitor or sensor at a predetermined time after the selection of the one or more user-selectable icons or buttons.

In accordance with one or more devices, systems, or methods of the present disclosure, the devices or systems may include an annunciator adapted to provide an alarm when data from a blood glucose monitor or sensor indicates a need to provide therapy.

In accordance with one or more devices, systems, or methods of the present disclosure, the systems or devices may include a processor and memory, the memory storing meal characterizations for each of the one or more user-selectable icons or buttons, and the processor being adapted to receive blood glucose data and update the meal characterizations for each of the one or more user-selectable icons or buttons based on the blood glucose data.

In accordance with one or more devices, systems, or methods of the present disclosure, the systems or devices may include memory that can store multiple meal characterizations for a single user-selectable icon or button based on the time of day.

In accordance with one or more devices, systems, or methods of the present disclosure, the systems or devices may include a cap that further includes a sensor adapted to detect a characterization of an insulin pen or a type of insulin in an insulin pen, a memory to store information about different types of insulin pens or different types of insulin, and a processor to determine the type of insulin pen or the type of insulin.

In accordance with one or more devices, systems, or methods of the present disclosure, the systems or devices may include a processor adapted to change the user interface dependent on the type of insulin pen or the type of insulin, where some types of insulin or insulin pens result in a user-interface that does not include any user-selectable icons or buttons adapted to announce a meal or an intent to have a meal.

### BRIEF DESCRIPTION OF THE DRAWINGS

FIG. 1A illustrates a hypothetical insulin delivery system that includes insulin pens, a glucose monitor or sensor, and a remote user interface device having a remote user interface.

FIG. 1B illustrates an exemplary meal announcement screen for a remote user interface device, which can be displayed by the remote user interface device of FIG. 1A.

FIGS. 2A and 2B depict an insulin pen with a first exemplary insulin pen cap having some of the features disclosed herein.

FIGS. 3A and 3B depict an insulin pen with a second exemplary insulin pen cap having some of the features disclosed herein.

FIG. 4 shows a schematic of internal components that may be included in the insulin pen caps of FIGS. 2A-3B.

FIGS. 5A and 5B depict an exemplary dose capture technique, which can be incorporated into the pen caps of FIGS. 2A-3B.

FIG. 6A is a flow chart of how a user may interact with an insulin pen cap provided herein.

FIG. 6B illustrates exemplary user interfaces that a user may see on a pen cap (or on an insulin delivery pen) during use of certain methods, systems, and devices provided herein.

FIG. 7 illustrates exemplary user interfaces that a user may see on a pen cap (or on an insulin delivery pen) when preparing certain methods, systems, and devices provided herein for use.

FIGS. 8A and 8B depict notices that may appear on a remote user interface device in some embodiments of methods, systems, and devices provided herein.

FIGS. 9A and 9B are charts illustrating how meal characterizations and user-specific dosage parameters can be initiated or updated.

FIG. 10A and 10B depicts how a user might transmit data between a flash glucose monitor and an insulin pen or insulin pen cap prior to administering insulin.

### DETAILED DESCRIPTION

Insulin delivery systems and devices, and methods for delivering insulin, can be designed to minimize the cognitive and active burden for people with diabetes (PWDs), or their caregivers, as they decide to administer insulin. In some embodiment's, methods, systems, and devices provided herein can passively capture diabetes-relevant data (e.g., insulin delivery data, blood glucose data, etc.) with or without providing the PWD (or a caregiver) with recommendations. In some embodiments, methods, systems, and devices provided herein can provide guidance regarding an appropriate dosage of insulin. In some embodiments, the dosage of insulin can be administered with an insulin delivery pen or syringe. In some cases, the insulin can be a long-acting insulin. In some cases, the insulin can be a quick-acting insulin. In some embodiments, an insulin delivery pen, or accessory therefor (e.g., a cap), can detect an amount of insulin delivered from the pen (or an amount of insulin that was set for delivery). In some cases, an insulin pen, or an accessory therefor, can include a user-interface, which can display data or recommendations to the user and/or permit the user to enter data into the insulin pen or accessory. The following exemplary system includes insulin delivery pens having dose capture pen caps, but alternative embodiments are also envisioned where the functionality disclosed herein is incorporated into other accessories for an insulin delivery pen or the insulin delivery pen itself.

### Exemplary Insulin Delivery System

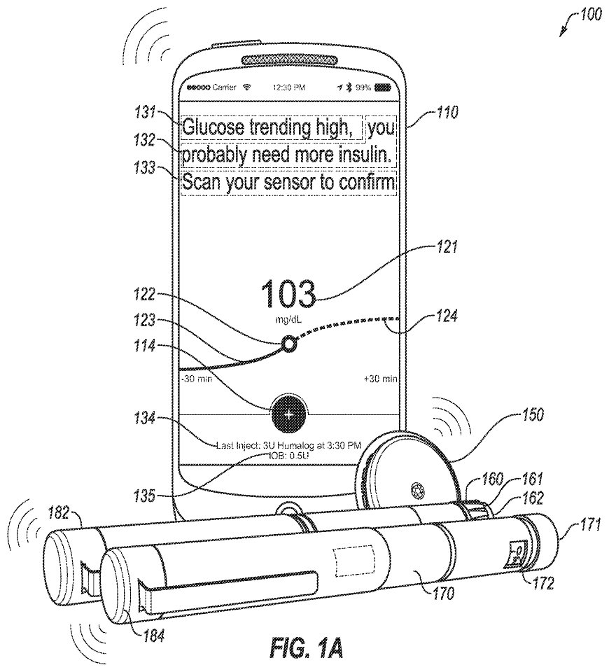

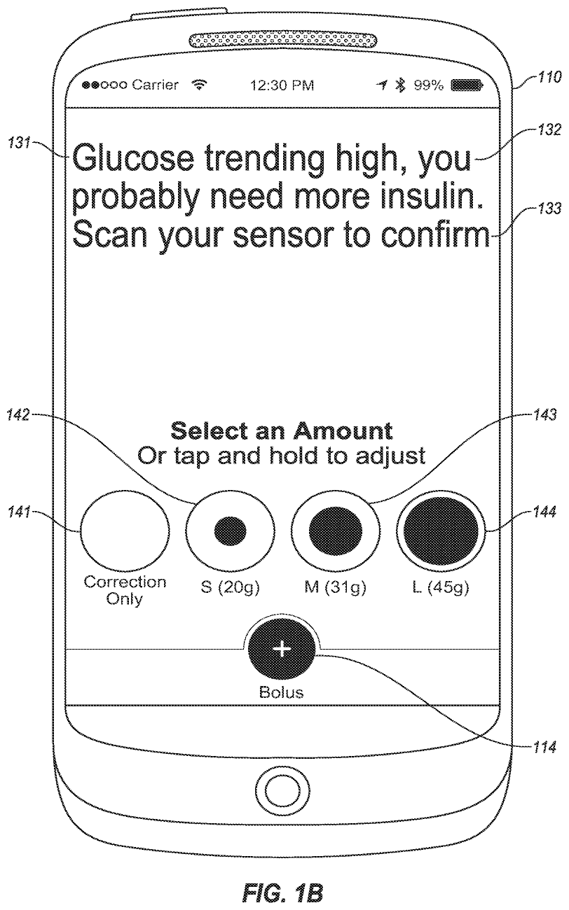

FIG. 1A illustrates a hypothetical insulin delivery system 100 that includes a quick-acting insulin (QAI) pen 160 (e.g., Humalog™, Novolog™, Apidra™), a long-acting insulin (LAI) pen 170 (e.g., Lantus™, Levemir™, Toujeo™, Tresiba™), a glucose monitor or sensor 150, and a remote user interface device 110. As shown, each insulin pen 160 and 170 includes a dose capture cap 182 and 184, respectively, which are in wireless communication with other components of system 100. As shown, the pens can include dials 161 and 171 for a user to set a dosage to be delivered and a dose indicator window 162 and 172. In alternative systems, the insulin pens 160 and/or 170 may themselves include dose capture technology and/or be in wireless communication with other components of system 100. Additional details about possible insulin pens and/or insulin pen caps is disclosed in greater detail below.

Glucose monitor or sensor 150 can be any suitable sensor device and/or monitoring system capable of providing data that can be used to estimate one or more blood glucose values. As shown, glucose monitor or sensor 150 can be a sensor configured to transmit blood glucose data wirelessly. For example, the glucose monitor or sensor 150 can include an optical communication device, an infrared communication device, a wireless communication device (such as an antenna), and/or chipset (such as a BLUETOOTH® device (e.g., BLUETOOTH® Low Energy, Classic BLUETOOTH®, etc.), a Near-field communication (NFC) device, an 802.6 device (e.g., Metropolitan Area Network (MAN), a ZIGBEE® device, etc.), a WiFi device, a WIMAX® device, cellular communication facilities, etc.), and/or the like. The glucose monitor or sensor 150 may exchange data with a network and/or any other devices or systems described in the present disclosure. In some cases, glucose monitor or sensor 150 can be interrogated with an NFC device by the user moving one or more components of the system near the glucose monitor or sensor 150 to power and/or transmit blood glucose data from the glucose monitor or sensor 150 to other components of system 100.

As shown, remote user interface device 110 is a smartphone, but any suitable remote user interface device can be used, such as a computer tablet, a smartphone, a wearable computing device, a smartwatch, a fitness tracker, a laptop computer, a desktop computer, a smart insulin pen (e.g., the dose capture caps 182 and/or 184), and/or other appropriate computing devices. As shown in the exemplary user interface of the exemplary mobile app running on the depicted smartphone, the user interface can include a bolus calculator button 114 and optionally other buttons for the user to enter data or to request recommendations. The exemplary user interface can also include a display of blood glucose data, past, present, or predicted. As shown, the user interface includes a graph of historical data from the previous 30 minutes 123, a continuation of that graph having projected data 124, a point indicator 122 showing the current (or most recent) estimated blood glucose value, and a display of the current (or most recent) estimated blood glucose value 121. The user interface can also include text explaining the glucose data 131, text providing suggested actions 132 and 133, such as text providing insulin, carbohydrates, or other therapy suggestions 132 and/or text suggesting that the user obtain blood glucose data 133. In some cases, the user interface can permit the user to click on the glucose data or otherwise navigate in the mobile app to obtain more detailed or more complete blood glucose data.

The user interface can also depict insulin data. In some cases, the user interface can indicate an amount of Insulin-on-Board (IOB) 135, which may be only for Quick-Acting Insulin. In some cases, an IOB calculation may be for both quick-acting and long-acting insulin. In some cases, the user interface can display the time and/or amounts of the most recent doses of quick-acting and/or long-acting insulins 134. In some cases, the user interface can permit the user to click on the insulin data or otherwise navigate in the mobile app to more detailed or more complete insulin delivery data. In some cases, a user interface can overlay blood glucose data and insulin delivery data in any suitable format, such as a graphical display of the timing of blood glucose data vs. the timing of insulin delivery data.

In use, a user (e.g., the PWD and/or a caregiver) can use system 100 to get recommendations regarding an appropriate insulin dosage. In the case of an upcoming need to deliver long-acting insulin, the message of 132 may change to provide a recommended long-acting insulin dosage. In some cases, a recommended dosage may appear on pen cap 184. In the case of the user wanting to deliver a bolus of quick-acting insulin, the user may press bolus calculator button 114 to enter into a bolus calculator. Although any suitable bolus calculator could be used in systems, methods, and devices provided herein, FIG. 1B depicts a possible user interface for a user to enter a meal announcement as either a correction only 141, a small meal 142, a normal sized meal 143, or a large meal 144. Upon selecting the meal size, the user interface can provide a recommended bolus dosage based on a number of carbohydrates associated with the corresponding button and optionally based upon blood glucose data.

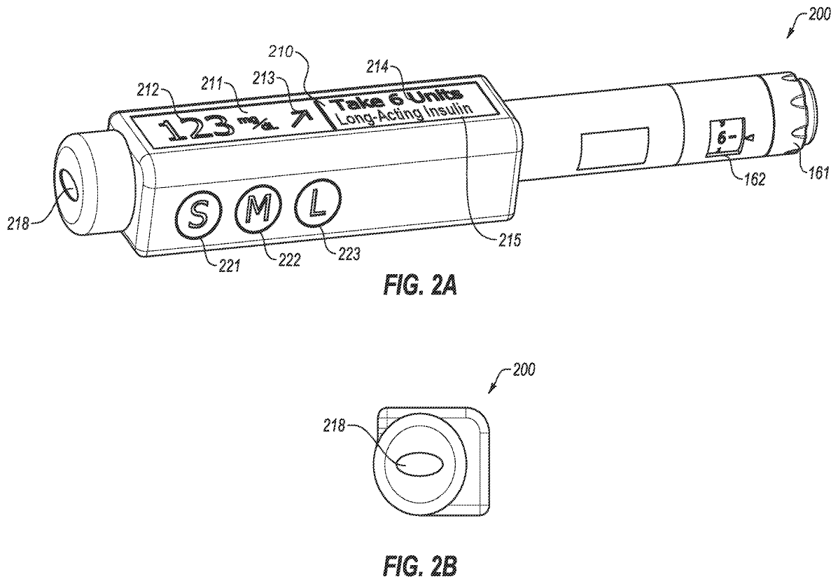

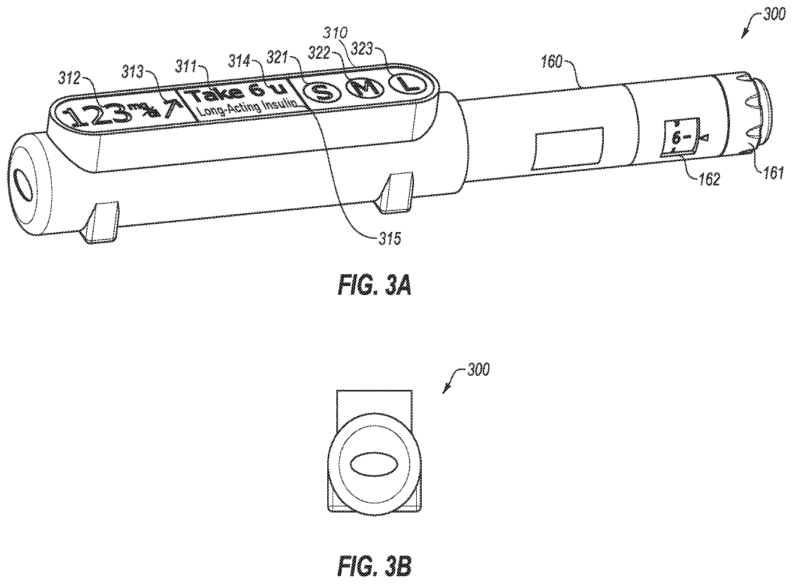

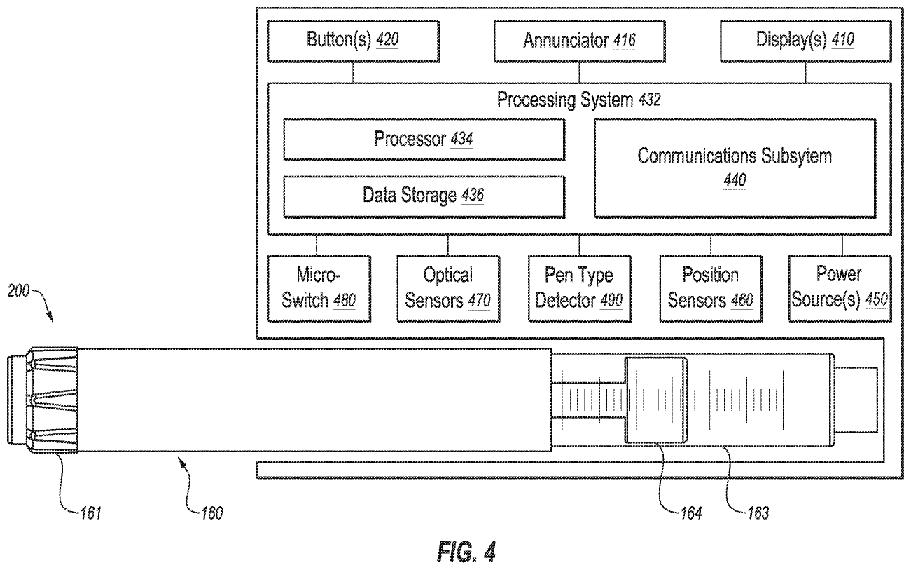

Additionally, or alternatively, dose capture pen caps for the quick-acting insulin pen 160 can include a user interface that permits the user to announce a small meal, medium meal, or large meal. FIGS. 2A-3B depict possible embodiments of dose capture pen caps 200 and 300 having buttons 221-223 and 321-323 that permit the user to announce a small (S), medium (M), or large (L) meal. In use, a user could announce whether the meal that they just ate or are about to eat is their normal meal size, (M), or larger (L), or smaller (S), and methods, devices, and systems provided herein can determine an appropriate bolus dosage of insulin based on the announcement and optionally based on blood glucose data, if available. Alternatively, an insulin pen can include dose capture technology and include a user interface provided herein and described as being on a dose capture pen cap. In some cases, a smart pen having dose capture capabilities can wirelessly communicate with a remote user interface (e.g., a smartphone).

### Personalizing Meal Announcement Buttons

As discussed above, methods, devices, and systems provided herein can provide a user with meal announcement buttons that provide the user with a reduced number of meal size selection options, which can be based upon the user's normal meal size, which can thus reduce the cognitive burden on a user seeking to administer insulin for a meal while improving the accuracy of insulin bolus recommendations. This section describes ways that methods, systems, and devices provided herein can determine an amount of insulin and/or an amount of carbohydrates to associate with each of the meal selection buttons (e.g., 141-144, 221-223, 321-323). Optionally, additional buttons can be present, such as a button that indicates a tiny meal or an extra-large meal for the user, such that any number of buttons are within the scope of the present disclosure. Additionally or alternatively, the system may include a single button, icon, or mode for announcing a meal to systems or devices of the present disclosure.

In some cases, each of the meal announcement buttons 142-144, 221-223, 321-323 can be associated with a number of carbohydrates that is personalized for the user based on other user-specific dosage parameters entered by the user for an insulin delivery system (e.g., total daily long-acting insulin dosage (e.g., U/day), a total daily dose of insulin (e.g., total of long and quick acting), a carbohydrate-to-insulin ratio, an insulin sensitivity factor, a glucose setpoint, or a combination thereof). In some cases, the number of carbohydrates assigned to each preset icon or button can be personalized over time based on estimations of the size of each meal consumed when that icon or button is selected based on a glucose response after the consumption of each meal. In some cases, the number of carbohydrates assigned to each preset icon or button can be rounded to the nearest 5 grams of carbohydrates and displayed. In some cases, a number of carbohydrates for each button is not displayed. In some cases, a user may manually enter personalized meal sizes for a number of user-selectable icons or buttons. In some cases, a number of carbohydrates assigned to each user-selectable icon or button can be initially set at a predetermined starting point or can be determined based on entered user information, and then iteratively adjusted upward or downward based upon the glycemic response to that selected meal size and bolus over time.

Initial settings for one or more meal announcement buttons 142-144, 221-223, 321-323 included on a device or in a system provided herein can be preset with predetermined values or ranges (e.g., small=20 g or 15-25 g, medium=30 g or 30-45 g, and large=50 g or 50-75 g). Additionally or alternatively, the initial settings can be set based on entered user data or based on one or more user-specific dosage parameters entered into a device or system provided herein. In some cases, initial settings for the one or more user-selectable icons or buttons can be based on an initially entered or determined and programmed total daily long-acting insulin (TDLAI) dose (e.g., U/day). For example, the relationship between the LAI dose [U/day] and Geometric Mean Meal Size [g] as characterized by the line corresponding to the major axis of the hyperellipsoid is: μ*MS=12.1*BRO.387. The relationship between Geometric Mean Meal Size [g] and Geometric Standard Deviation Meal Size is: σ*MS=1.92−μ*MS/186 where MS may represent the meal size and BR may represent the basal rate of insulin. Accordingly, initial meal size groups may correspond to predetermined percentiles of the Meal Size distribution by combining the above equations, optionally rounding meal size groups to the nearest 1, 5, or 10 grams. In some cases, the relationship between typical meal sizes and other user-specific dosage parameters can be determined according to population statistics. In some cases, the number of carbohydrates associated with each user-selectable icon or button can be displayed on and/or adjacent to the user-selectable icon or button, which can help a user understand how to use the insulin delivery device or system to avoid deskilling the user. For example, seeing the number of carbohydrates assumed for each meal size helps a user that thinks about meals in terms of carbohydrates to adjust to using buttons to indicate a size of a meal. Additionally, by starting with display numbers rounded to the nearest 5 grams, the user can perceive that precision is not required, thus also reducing the cognitive burden on the user. Additionally, as the system iterates to personalize the amount of carbohydrates for each particular user-selectable icon or button, the system can adjust these numbers by smaller units (e.g., by 1 gram) to demonstrate to the user that the system is adjusting the number of carbohydrates associated with user-selectable icon or button.

In some cases, the user interface may be configured such that a PWD may interact with the user interface to enter more detailed information regarding the bolus size outside of the default options. For example, a PWD may be presented with a series of pre-set sizes that are readily adjustable in increments of 5 g by selecting a size and scrolling up or down. By interacting further with the user device (e.g., pressing and holding on the meal size), the user may have the option to manually input a bolus size or adjust the size in increments of 1 g.

Methods, systems, and devices provided herein can update the number of carbohydrates associated with each user-selectable icon or button using any suitable method. In some cases, methods, systems, and devices can use postprandial blood glucose data (e.g., between 1 hour and 3 hours after an announced meal) to evaluate whether the PWD likely consumed significantly more or significantly less carbohydrates than programmed for the user-selectable icon or button. In some cases, one or more postprandial blood glucose thresholds can be used to evaluate the match between the amount of carbohydrates consumed and the amount of carbohydrates associated with a selected user-selectable meal icon or button. For example, methods, devices, and systems provided herein can ask a user for a postprandial blood glucose reading from a glucose sensor, glucose monitor, or blood glucose meter. In some cases, a glucose sensor can be a flash glucose monitor and methods, systems, and devices provided herein can prompt the user to interrogate the flash glucose monitor at a predetermined postprandial time period. As used herein, the term “flash glucose monitor” may refer to a device configured to provide blood glucose readings in response to a manual invocation of the device, typically by a physical signal (e.g., a button, tap, etc.) or a wireless signal (e.g., a near-field communication (NFC), BLUETOOTH® communication, etc.). Such blood glucose readings may be performed periodically and reported when the device is invoked, or may be taken when invoked. In some cases, methods, devices, and systems provided here can receive postprandial blood glucose data from a continuous glucose monitor. In some cases, methods, systems, and devices provided herein can use a single postprandial blood glucose data point and compare that to one or more upper thresholds and one or more lower thresholds for that period of time to determine whether the number of carbohydrates associated with that user-selectable meal icon or button should be adjusted upward or downward. For example, if a user selects a typical meal icon indicating a meal of 30 grams of carbohydrates, but the 2-hour postprandial blood glucose reading is above 200 mg/dL, the number of grams associated with that icon or button might be adjusted upward by 2 grams, if it is above 170 mg/dL, it might be adjusted upward by 1 gram, if it is below 130 mg/dL, it might be reduced by 1 gram, and if it is below 100 mg/dL, it might be reduced by 2 grams. Accordingly, over time the meal icons would be adjusted to more closely resemble the user's typical consumption patterns in a way that matches the user's mental model surrounding the meals that they eat. The particular thresholds can be determined based on the postprandial time, the number of grams associated with the meal icon or button, the CR, ISF, and daily dose of LAI, and setpoint of the PWD, etc.

In some cases, meal announcement buttons can be personalized based on the time of the day. For example, in some cases, a user may have a larger average dinner and smaller average morning meals, and methods, devices, and systems provided herein can estimate an amount of carbohydrates for a user based on the time of day. In some cases, the amount of carbohydrates and the meal sizes (S, M, L) can be displayed together to help a user understand that the personalization is specific to the user's daily pattern. In some cases, buttons can be personalized based on the day of the week (e.g., a user's weekend meal patterns might be significantly different than during weekdays).

Because diabetes is a highly personal disease that presents the PWD or their caregiver(s) with significant cognitive burdens surrounding the determination of appropriate dosages of insulin, some PWDs or caregivers develop their own techniques (or mental model) for estimating an appropriate dosage of insulin. Although methods, systems, and devices provided herein can be adapted to provide recommendations to a user, the user may be free to dose insulin according to the user's preferences and the user's specific knowledge of what the PWD is about to eat and/or is experiencing (e.g., exercise, sickness, etc.). In some cases, meal announcement buttons can change based on repeated patterns of a user administering doses of insulin above or below a recommended dosage of insulin so that the meal announcement buttons begin to match the user's mental model regarding a typical meal size. Adjustments to an amount of carbohydrates represented by each meal announcement button based on the actual dosage, however, may be determined based on the postprandial blood glucose readings of a PWD. For example, if the postprandial blood glucose readings indicate an appropriate dosage, it can indicate that the user's mental model is appropriate for that meal, and that the system can thus adjust the meal announcement buttons to match the user's mental model (e.g., reduce the size of the meal assumed for a (S) meal based on a repeated pattern of the user administering less insulin than recommended for an (S) meal selection if postprandial blood glucose readings are usually within a predetermined range). However, in some cases, methods, devices, and systems, provided herein can use postprandial blood glucose readings to determine if the user's mental model failed to determine the appropriate dose. In some cases, a high or low postprandial blood glucose reading can prevent methods, systems, and devices provided herein from adjusting the meal announcement buttons based on meals where one or more postprandial blood glucose readings indicate a mismatch between the dose and the meal. For example, if a user administers less insulin than recommended for a selected small meal and has one or more high postprandial blood glucose readings, methods, systems, and devices provided herein can ignore that administration for the personalization of the meal announcement buttons. In some cases, methods, systems, and devices provided herein can provide notices to a user if the user is consistently ignoring the recommended dosages in a way that causes the PWD to go high or low after a meal if the usage pattern indicates a mismatch between the user's mental model and the PWD's physiology and food consumption patterns. For example, if the user is consistently administering less insulin than recommended and consistently having high blood glucose readings after a meal, a notice may indicate to the user that the user should consider administering the recommended doses at meal times in order to achieve better glycemic control. Accordingly, in some cases, methods, systems, and devices provided herein can be designed to improve the match between the user's mental model and the PWD's physiology and food consumption patterns.

In some cases, a remote user interface device 110 can permit a user to manually enter a specific number of carbohydrates into a bolus calculator for a recommendation for a specific meal. In some cases, methods, systems, and devices can use repeated patterns of a user requesting the same meal size recommendation to update the size of a meal announcement button or to add another meal announcement button.

### Dose Capture Pen Caps

FIG. 1A depicts a system that can include dose capture pen caps 182 and 184, which can transmit data to and/or from a glucose monitor or sensor 150 and/or to/from a remote user interface device 110. The pen caps in FIG. 1A may or may not include a user interface. Pen caps 182 and 184 can use any suitable technology to determine an amount of insulin that has been administered from insulin pens 160 and 170. In some cases, not shown, insulin pens 160 and 170 can include dose capture technology and can communicate wirelessly with the remote user interface device 110 and/or glucose monitor or sensor 150.

FIGS. 2A-3B depict alternative embodiments of pen caps, 200 and 300, which can be used with an insulin pen to assist a PWD or caregiver (the user) with dosing decisions.

FIGS. 2A and 2B depict an embodiment of a pen cap 200 that includes a display screen 210 that can display an estimated glucose value (EGV) 212, the units for the EGV 211, and a trend indicator for the EGV 213. The display can also provide a recommended dosage 214 and an identification of the type of insulin 215. Pen cap 200 can also include meal announcement buttons 221-223. Additionally, the display may also indicate the time and amount of the previous dosage and/or an IOB value to remind a user about their most recent dosage. Although three meal announcement buttons are shown, in some cases a pen cap can include no meal announcement buttons and meals can be announced on a remote user interface device, such as device 110 as depicted in FIG. 1A. In some cases, pen cap 200 can include a single meal announcement button. In some cases, pen cap 200 can include between 2 and 6 different meal announcement buttons. In some cases, pen cap 200 can include a correction only button. Pen cap 200 can also include one or more indicator lights, such as indicator light 218, which can light up to indicate that it is transferring data, light up to indicate that the user's attention is needed, and/or light up to indicate whether a dose capture functionality is or is not working.

FIGS. 3A and 3B depict another embodiment of a pen cap 300 that includes a touch screen user interface 310 that can include meal announcement buttons 321-323 and display an estimated glucose value (EGV) 312, the units for the EGV 311, a trend indicator for the EGV 313, a recommended dosage 314 and an identification of the type of insulin 315. Additionally, the display may also indicate the time and amount of the previous dosage and/or an IOB value to remind a user about their most recent dosage. Although three meal announcement buttons are shown, the touch screen user interface 310 of pen cap 300 can be customized based on the user's preferences and/or the type of insulin to display different numbers of meal announcement buttons 321-323 and/or to not include meal announcement buttons. For example, if pen cap 300 is placed on a long-acting insulin pen, it may be capable of detecting the type of insulin and automatically updating the display to correctly identify the type of insulin at 315, but also to remove the meal announcement buttons 321-323 and replace them with other content.

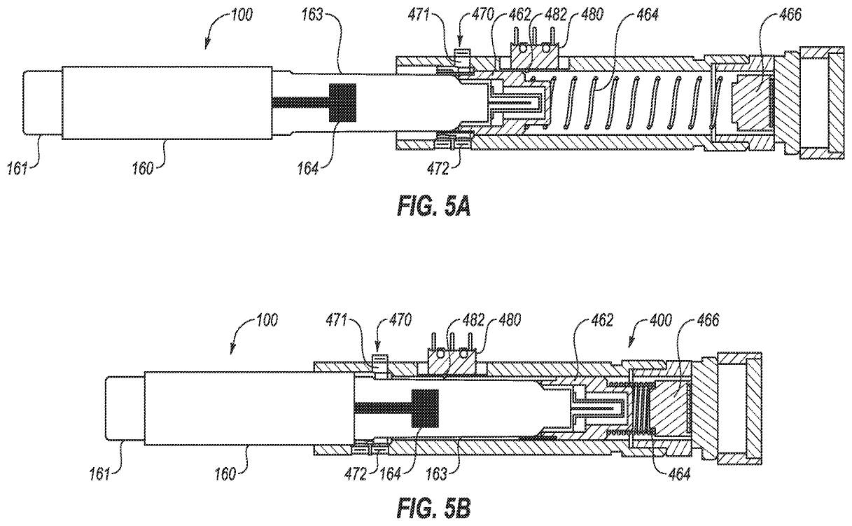

Although pen caps 182, 184, 200, and 300 can use any suitable technology to estimate an insulin dosage, FIGS. 4, 5A, and 5B depict exemplary embodiments of pen cap components that may be used to detect a dosage of insulin.

FIG. 4 depicts a representation of the internal components of an exemplary pen cap 400. As shown, the pen cap 400 can include one or more displays 410, one or more buttons 420, and one or more annunciator(s) 416, each controlled by a processing system 432. Processing system 432 includes a processor 434, data storage (or memory) 436, and a communications subsystem 440. The communications subsystem 440 can enable wireless communication between the pen cap and a remote user interface device (e.g., 110 from FIG. 1A) or a glucose sensor or monitor (e.g., 150 from FIG. 1A). In some cases, the communications subsystem 440 can include a near field communications (NFC) chip. In some cases, the communications subsystem 440 can include a BLUETOOTH® Low Energy (BLE) chip. In some cases, the communications subsystem 440 can include an optical communication device, an infrared communication device, a wireless communication device (such as an antenna), and/or chipset (such as a BLUETOOTH® device (e.g., BLUETOOTH® Low Energy, Classic BLUETOOTH®, etc.), a Near-field communication (NFC) device, an 802.6 device (e.g., Metropolitan Area Network (MAN), a ZIGBEE® device, etc.), a WiFi device, a WIMAX® device, cellular communication facilities, etc.), and/or the like. In these and other cases, the communications subsystem 440 can exchange data with a network and/or any other device or system described in the present disclosure. The pen cap 400 includes a power source 450, which may be a rechargeable or non-rechargeable battery. The processing system can determine a pen type from data from a pen type detector 490. The processing system can also determine a position of a plunger within an insulin delivery pen using one or more optical or position sensors and/or micro-switches, such as micro-switch 480, optical sensor(s) 470, and position sensor(s) 460. FIGS. 5A and 5B illustrate the arrangement of a position sensor 460, micro-switch 480, and optical sensor 470 within a pen cap 400. The optical sensor 470 can include a light 471 and photoreceptor 472 positioned on opposite sides of an insulin delivery pen so that light passes through the insulin vial 163 and received by the photoreceptor 472 until the plunger 164 of the insulin pen 160 passes by the optical sensor 470. The tip of insulin pen 160 is received by a slider 462 that slides within the pen to trigger a micro-switch 480 having trigger 482 to tell the pen cap to use optical sensor 470 to identify when the plunger 164 passes the optical sensor 470. Data from position sensor 460, which includes spring 464, slider 462, and proximity sensor 466 to determine the distance that the insulin pen in inserted into the pen cap when the plunger passes the optical sensor 470 and thus determine an amount of insulin remaining in insulin vial 163. Data from prior use of the pen cap 400 can then be used to estimate an amount of insulin delivered to the patient. More detail about how this dose capture technique can be used is disclosed in PCT Publication WO 2017/009724 A1, which is hereby incorporated by reference in its entirety.

### Exemplary Use of Dose Capture Device/System

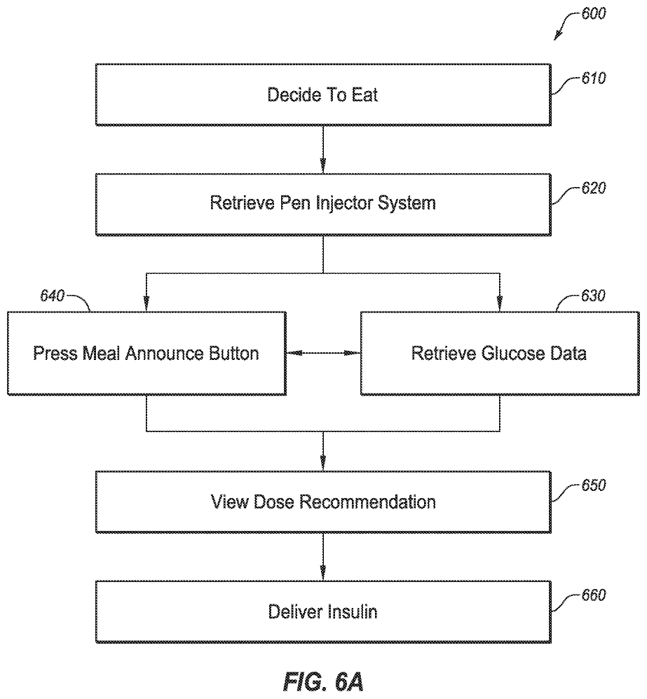

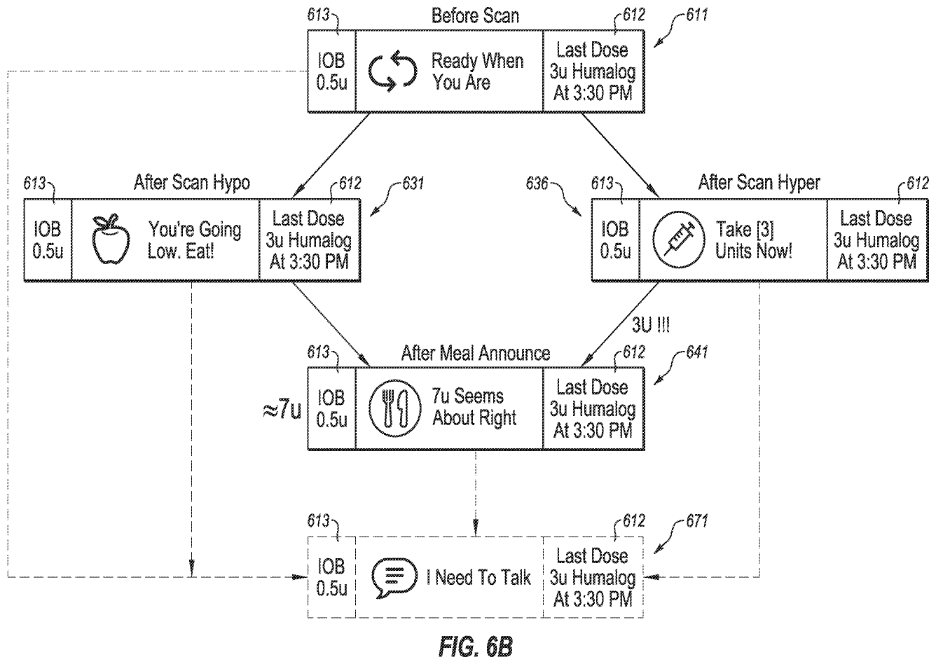

FIG. 6A is a flow chart depicting how a user may decide, when eating, to dose insulin using methods, systems, and devices provided herein. As show, process 600 starts with the user deciding to eat something (or deciding to bolus for food already eaten) in step 610, followed by the user retrieving a pen injector in step 620. After retrieving a pen injector, the user may decide to announce a meal in step 640 and/or retrieve glucose data in step 630, which can occur in either order. After announcing a meal and/or retrieving glucose data, the user can view a dosage recommendation, which might appear on a pen cap or may appear on a remote user interface device, in step 650. After viewing the recommendation, the user can then decide how much insulin to deliver and deliver the insulin in step 660.

FIG. 6B depicts an exemplary user interface displays for a pen cap for the user when the user is following process 600. As shown, user screen 611 can indicate that the system is active and display data about a most recent dose 612, such as the type, amount, and time of day, and/or an IOB value 613. If a user obtains a blood glucose value in step 630 that indicates a hypoglycemic condition, user screen 631 can indicate that the user should consume carbohydrates, and may continue to display information about a recent dosage and/or an estimated IOB. If a user obtains a blood glucose value in step 630 that indicates a hyperglycemic condition, user screen 636 can indicate that the user should dose insulin and provide a recommended dosage, and may continue to display information about a recent dosage and/or an estimated IOB. In some cases, the recommended dosage may indicate an amount that a user should add to a dosage that they would otherwise take for a meal. If a user then enters a meal announcement in step 640, user screen 641 can appear and indicate a recommended insulin dosage to account for a meal and the current EGV. User screen 671, however, may appear at any time when the pen cap detects that something has gone wrong or otherwise requires the user's attention. In some cases, pressing buttons on a pen cap can cause the user screen on the pen cap to display more information and/or the user screen 671 can direct the user to troubleshoot issues with the system on a remote user interface device, such as remote user interface device 110 in FIG. 1A.

When a user administers insulin in step 660, the amount of insulin administered may differ from a recommended dosage and/or the recommendation may simply be that the user adjust their mental model of how much to administer for a meal, for example if the user does not make a meal announcement and doses insulin based on user screen 636. As shown in user screen 636, the dosage recommendation may be indicated in brackets to indicate an amount of insulin that should be used to correct for an elevated blood glucose level, and thus the user can add that to the amount of insulin that the user would ordinarily administer for a meal. Accordingly, methods, systems, and devices provided herein can infer the amount of carbohydrates eaten for a meal in the user's mental model based on an amount of insulin delivered by the user. For example, if a user retrieves an EGV from a glucose sensor or monitor in step 630 and sees a recommendation to take 3 units of Humalog in addition to what they would normally take, and then doses 10 units of Humalog, then the methods, systems, and devices provided herein can infer that the user ate a meal and estimate the size of the meal based on the bolus size. The estimated meal size can then be used by the system to further personalize meal announcement buttons (e.g., buttons 142-144, 221-223, and 321-323) and user-specific dosage parameters. Additionally, in some cases a user will announce a meal, but administer an amount of insulin that differs from the amount recommended, in which case methods, systems, and devices provided herein can either ignore the postprandial data for that administration for personalizing the meal announcement buttons (e.g., buttons 142-144, 221-223, and 321-323), but perhaps use the postprandial data for updating user-specific dosage parameters. For example, if a user is about to eat a meal that is between the user's mental model for a medium sized meal and the user's mental model for a large meal, the user might retrieve an EGV (e.g., in step 630) and look at a screen similar to user screen 636 to find out the amount for the correction dosage, and then announce a meal (e.g., in step 640) as a medium meal to see a screen similar to user screen 641 and then announce a meal again (e.g., conduct step 640 again) as a large meal to see a different recommendation, and then the user might deliver an amount of insulin between the two recommendations. Methods, systems, and devices provided herein can use data from a dose capture technique to estimate an amount of insulin actually delivered and use that insulin delivery data to determine an estimated size of each meal, regardless of whether the user announces the meal or follows the recommendation. Additionally, variations from the recommendations and postprandial glucose data can be used to determine adjustments to a number of carbohydrates represented by each meal announcement button e.g., buttons 142-144, 221-223, and 321-323) so that they match the user's mental model, as discussed above.

In many cases, a user will use their own mental model for administering boluses of insulin for meals and only use the system to determine a correction dose after obtaining an EGV (e.g., in step 630) and viewing a screen similar to user screens 631 or 636, which can in some cases indicate a correction dose only or might display two recommendations, (a) an amount to dose or suggestion to eat if the user is only seeking to correct a hyperglycemic or hypoglycemic condition and (b) a change to how much the user would typically dose if the user is eating. In some cases, the calculations can use different equations based on reducing a risk of a hypoglycemic condition. In some cases, a calculation for an amount to change the user's typical dose of insulin if the user is eating can incorporate adjustments based on detected patterns of the user over or under dosing insulin for meals in order to adjust for detected mismatches between the user's mental model and the PWD's physiology and food consumption patterns.

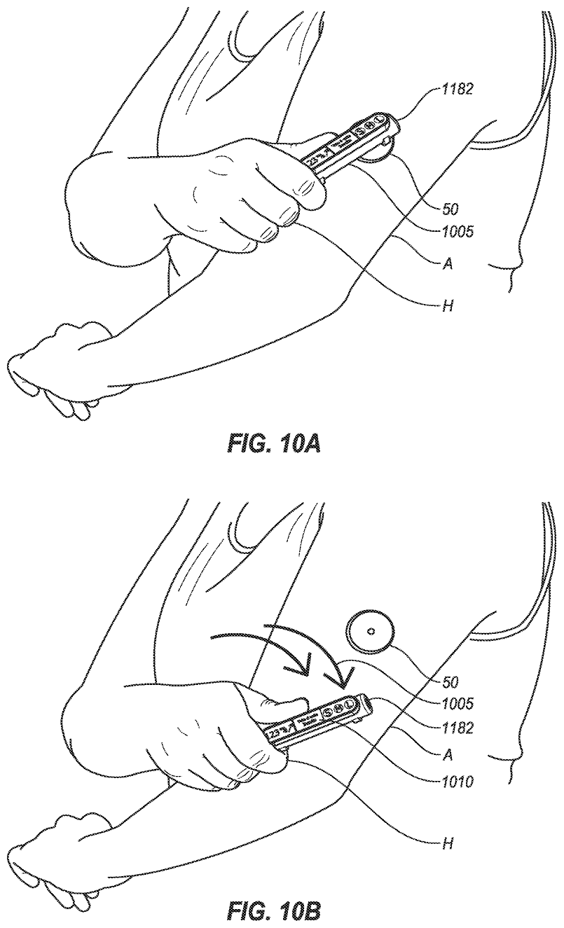

FIGS. 10A and 10B depict exemplary systems for how a user can retrieve blood glucose values from a glucose sensor that can communicate using near field communications by swiping 1005 a pen cap near the glucose sensor 50, optionally with swiping motion 1005. The cap can include a near field communication chip 1182. After swiping 1005, display 1010 can appear on the pen cap. Wireless Communications and the Pairing Process

Referring back to FIG. 1A, systems provided herein can include one, two, or more pen caps 182 and 184 (or alternatively pen caps 200 and 300 in FIGS. 2A-3B), a remote user interface device 110 (e.g., a smartphone), and a glucose sensor or monitor 150 (e.g., a flash glucose monitor, a continuous glucose monitor, a blood glucose meter), which can all be in wireless communication with each other. In some cases, the glucose sensor or monitor 150 can be a flash glucose monitor adapted to communicate with the pen caps 182 or 184 via near field communication. In some cases, the glucose sensor or monitor 150 can be a continuous glucose monitor adapted to communicate with the pen caps 182 or 184 via radio signals, such as radio signals using BLUETOOTH® Low Energy protocols. Additionally or alternatively, such communication may occur over NFC, WiFi, ZIGBEE®, Classic BLUETOOTH®, or any other communication protocol, device, or technique. In some cases, a flash glucose monitor or a continuous glucose monitor can require a pairing process in order to communicate with other devices and/or can require a warm up period before it is ready to be used. For example, a broadcasting device (which may be either (1) the flash glucose monitor/continuous glucose monitor, glucose sensor or monitor 150 or (2) the pen caps 182 or 184 or other processing device in accordance with the present disclosure) may broadcast a pairing signal that is received by the other device, after which a series of data exchanges or handshakes may occur to establish a secure communication session between the two devices. In some cases, such a pairing procedure may be facilitated by invoking one or more of the buttons of the pen caps 182 or 184. Additionally or alternatively, the pairing procedure may be invoked using a user interface device paired or otherwise coupled with one or both of the glucose sensor or monitor 150 or the pen caps 182 or 184.

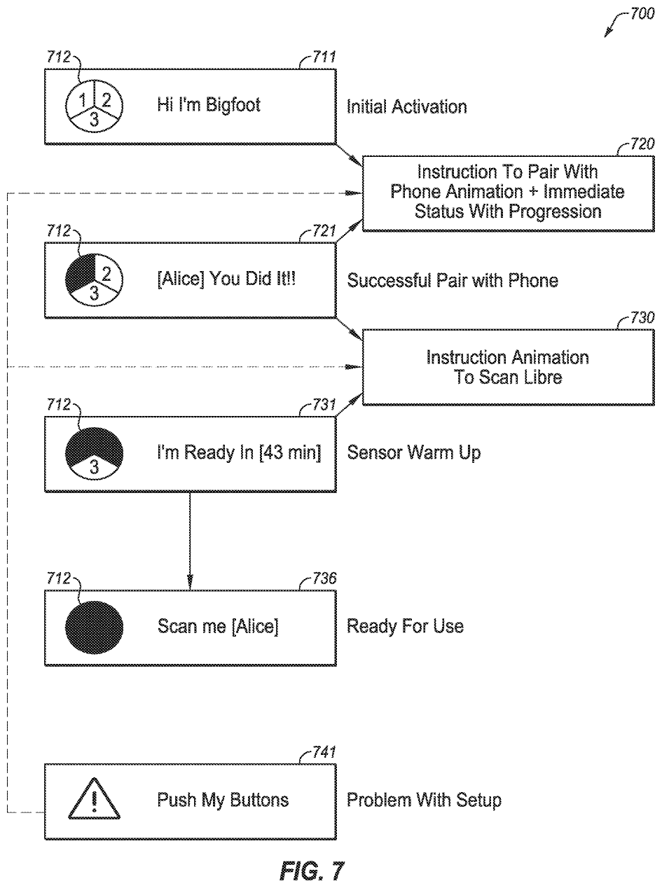

FIG. 7 illustrates an exemplary process 700 of starting the use of a system, such as the system depicted in FIG. 1A. In user screen 711, a pen cap can include a welcome screen including a connection indicator 712 that indicates that it has not been paired. Connection indicator 712 can include segments that indicate steps that must be completed before the system can be used. In some cases, a screen can be on a remote user interface device (e.g., remote user interface device 110 from FIG. 1A) indicating how the user should pair the pen cap to the remote user interface device. In step 720, the user can receive instructions to pair a remote user interface device (e.g., a smartphone) with the pen cap on the remote user interface device or the pen cap. After the successful pairing of the remote user interface device with the pen cap, screen 721 can appear to indicate the successful pairing with a remote user interface device. Subsequently, in step 730, instructions for connecting the pen cap and/or the remote user interface to a glucose sensor or monitor 150 can appear on the pen cap or the remote user interface. In some cases, the glucose sensor or monitor 150 can be a flash glucose monitor using near field communications, and methods devices, and systems provided here can instruct the user to create a near field communication link between the glucose sensor or monitor 150 and the remote user interface device and/or with the pen cap 182 or 184 to establish a communication link. In screen 731, the pen cap user screen can indicate that a sensor or monitor is warming up, possibly with a countdown clock. In screen 736, the pen cap user screen can indicate that the glucose sensor or monitor 150 is ready for use (after the warm up period has completed). Screen 741 can also appear at any time during the use of the system or during the pairing process to indicate that the pairing has failed, and then return the user to step 720 or 730 to fix the pairing issue. For example, glucose sensors and monitors can have a use life (e.g., 3 days, 7 days, 10 days, 14 days) and require replacement after the use life, so screen 741 may appear after the glucose sensor or monitor has expired, and the user can view instruction for replacing the sensor or monitor and connecting the system to that new glucose sensor or monitor in a step 730. Also, if there is a problem in communicating to a remote user interface device, a screen 741 can appear and bring the user back to step 720 to connect a new remote user interface device or to reconnect the remote user interface device.

### Passive and Active Information Gathering

Methods, devices, and systems provided herein can be adapted to gather information about insulin usage and user eating patterns passively without requiring the user to perform extra steps, but be available to help a user determine appropriate actions when called upon by the user. As such, methods, systems, and devices provided here can use dose capture technology in or attached to insulin pens to estimate amounts of insulin delivered to the person with diabetes (PWD). Additionally, estimated blood glucose values (EGVs) can be pushed or pulled to the pen caps and/or to the remote user interface device though wireless communications as discussed above, and be available to the user to help the user make insulin delivery decisions. In some cases, the glucose sensor or monitor 150 can be a flash glucose monitor that requires user interaction to retrieve an EGV. In some cases, a system including a flash glucose monitor can receive both a current EGV and past EGVs from an interrogation of the flash glucose monitor, which can be used by methods, devices, and systems provided herein to make therapy recommendations and to update user-specific dosage parameters.

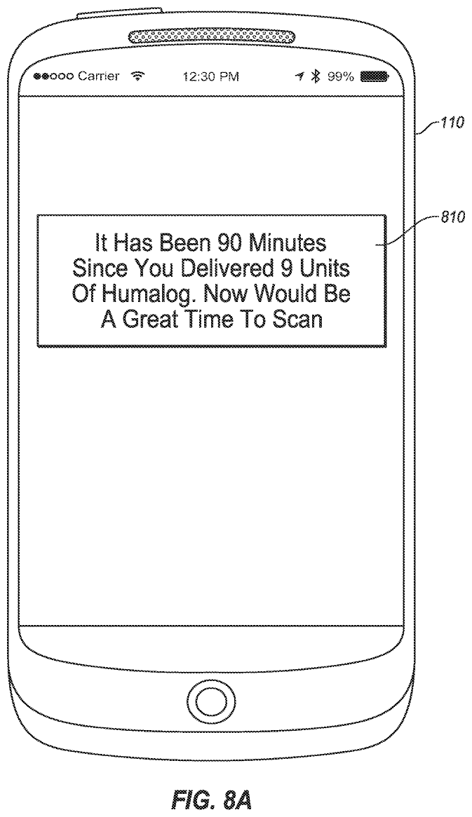

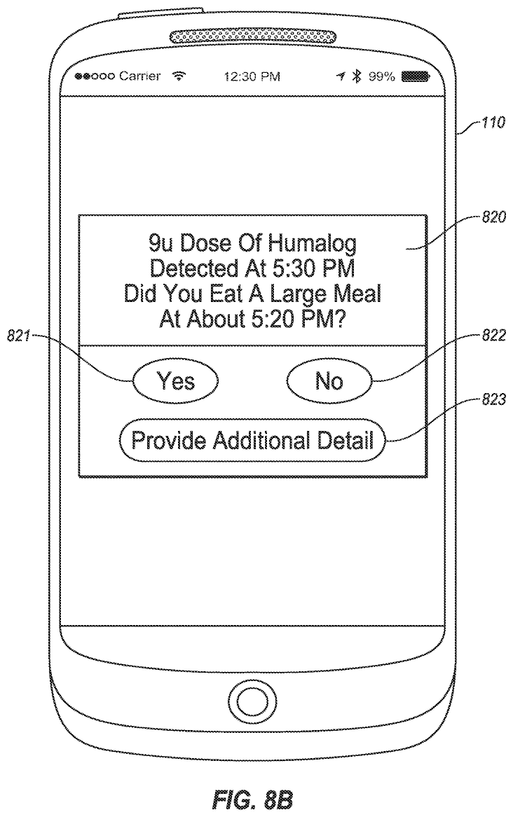

In some cases, methods, devices, and systems can include user prompts to request information from the user or to request that the user obtain an EGV, based on risks to the user and/or to obtain data. For example, as discussed above, postprandial data can be used to update the meal announcement buttons. Additionally, postprandial data can be used to update other user-specific dosage parameters. Moreover, after a meal, a user is at an elevated risk of having a hyperglycemic or hypoglycemic condition. Accordingly, in some cases, methods, systems, and devices provided herein can request a user obtain an EGV. For example, in FIG. 8A, a remote user interface device 110 can include a notice 810 displayed at 90 minutes after the administration of a bolus dose of insulin in order to encourage a user to scan a flash glucose monitor. Alternatively, notice 810 can ask the user to obtain blood glucose data from any other suitable glucose monitor or sensor (e.g., from a blood glucose meter requiring a finger stick, etc.). Alternatively, notice 810 can appear on a pen cap or a smart insulin pen. By obtaining the postprandial blood glucose data, methods, systems, and devices provided here can (a) determine whether correction doses may be prudent and/or (b) determine how to personalize meal announcement buttons.

Methods, devices, and systems provided herein can also seek feedback from a user regarding the user's mental model, especially if a user fails to announce a meal size. For example, in some cases, a user determining a dosage of quick-acting insulin may follow the steps shown in FIG. 6B, but stop short of announcing a meal, but instead just bolus for a meal after seeing a suggested correction dose in user screen 636. As discussed above, methods, systems, and devices provided herein can estimate a number of carbohydrates for a meal based on a difference between a recommended correction dosage and an actual dosage. In some cases, methods, devices, and systems provided herein can seek confirmation about an estimated number of carbohydrates from the user when the user accesses a remote user interface. For example, FIG. 8B depicts a message 820 that might appear on a remote user interface device 110 asking the user to confirm by selecting YES 821 or deny by selecting NO 822 whether the PWD ate a particularly sized meal. In some cases, message 820 can appear after blood glucose data is retrieved. In some cases, message 820 can use multiple blood glucose values to determine the likely timing of the meal and the likely size of the meal, which can differ from an amount of carbohydrates inferred based on the insulin size. Additionally or alternatively, methods, systems, and devices of the present disclosure may continue with analysis and/or data collection with an estimated meal size of the PWD even without input from the PWD regarding the size of the meal or even whether or not the PWD consumed a meal. In some cases, a difference between an estimated meal size based on postprandial blood glucose values and insulin delivery data and an estimated meal size based on insulin delivery data alone can indicate a mismatch between the user's mental model and the PWD's physiology and food consumption. In some cases, message 820 can be passive (i.e., without an audible alert), but be available for a user to answer when the user looks at the remote user interface device or opens a mobile app for the system on the remote user interface device. In some cases, message 820 can provide insights showing the user that the methods, systems, and devices understand the user's usage patterns in order to build the user's trust in the devices and systems. In some cases, a user may select button 823 to provide additional details about the bolus. Data received from the user after a meal can then be used to make updates to the meal announcement buttons 142-144, 221-223, 321-323.

### Calculating and Updating Recommendations, User-Specific Dosage Parameters, and Active Insulin

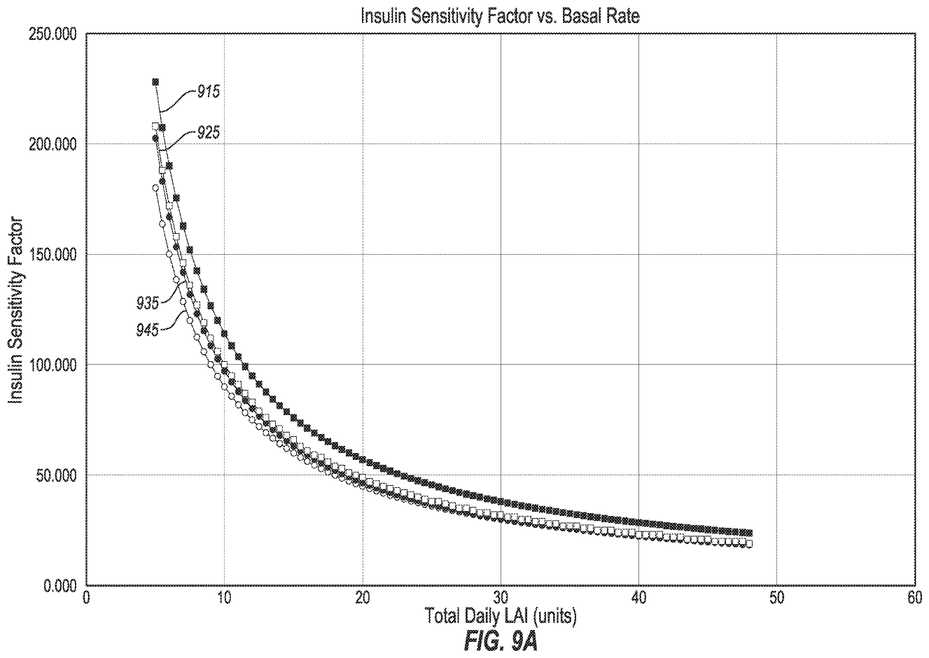

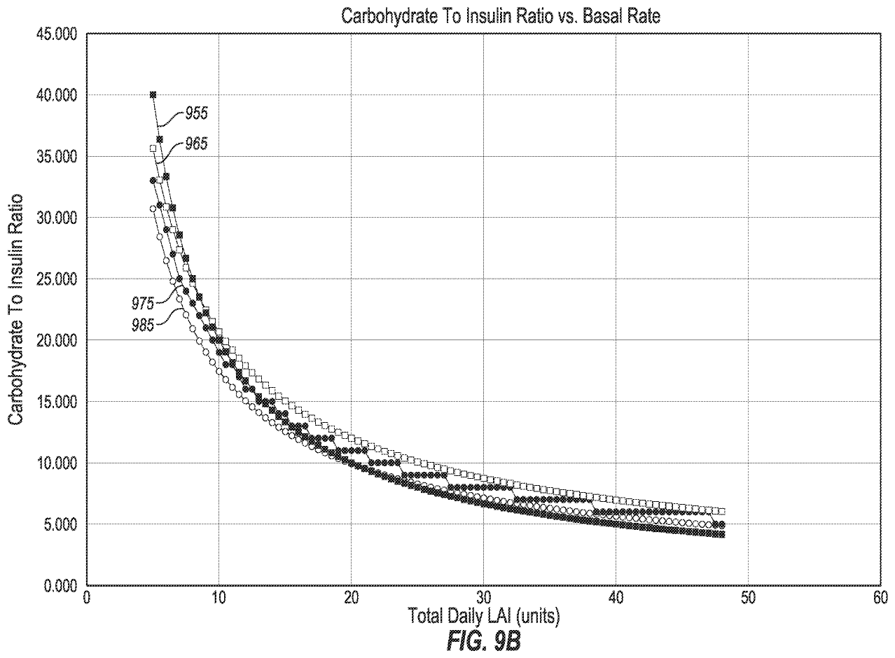

Methods, devices, and systems provided herein can use any suitable technique for making recommendations, for updating user-specific dosage parameters (e.g., the person's ISF, CR, Total Daily LAI dosage, etc.), and for estimating amounts of unacted insulin (e.g., for calculating IOB). In some cases, the user-specific dosage parameters can vary depending on the time of day. In some cases, the user-specific dosage parameters can be determined using a fixed relationship between the user-specific dosage parameters. For example, in some cases such as a user having Type 1 Diabetes, a fixed relationship between Total Daily LAI and the PWD's carbohydrate-to-insulin ratio and the PWD's Insulin Sensitivity Factor can be based on fixed mathematical relationships. In some cases, the relationships may be determined by one of the plotted lines 915, 925, 935, 945, 955, 965, 975, or 985 shown in FIGS. 9A and 9B, or any plotted line between lines 915 and 945 and between lines 955 and 985. By having a fixed mathematical relationship between Total Daily LAI and ISF and CR, methods, systems, and devices provided herein can update CR and ISF as a PWD's response to insulin changes over time.

Methods, systems, and devices, can, in some cases, make recommendations to the user to adjust dosages of LAI based on fasting blood glucose readings (e.g., blood glucose readings taken in the morning before the PWD has eaten). In some cases, methods, devices, and systems provided herein can increase by a set number of units (e.g., 0.5 units) based on fasting blood glucose readings being above a threshold and decrease the recommended dosage of LAI based on fasting blood glucose readings being below a different lower threshold. In some cases, methods, devices, and systems can provide recommendations regarding a dosage of LAI and quick-acting insulin (QAI) for if the user fails to deliver the LAI at an appropriate time.

Methods, devices, and systems provided herein can calculate a recommended correction bolus by subtracting a target blood glucose value from the EGV and dividing that number by the Insulin Sensitivity Factor (ISF) and then subtracting the IOB. Methods, devices, and systems provided herein can also calculate a recommended bolus for food consumption by dividing a number of carbohydrates associated with a food announcement button by a carbohydrate-to-insulin ratio (CR) stored for the PWD. Conversely, if a PWD delivers a bolus of insulin after calculating a correction bolus without entering a meal and that bolus differs from the recommended correction bolus, methods, systems, and devices provided herein can calculate an amount of inferred carbohydrates for the meal by subtracting the recommended correction bolus from the amount of insulin delivered and multiplying that number by a CR stored for the user. In other words, Inferred Carbs=(Bolus of Insulin delivered−Recommended Correction Bolus)*CR. Methods, systems, and devices provided herein can then use the calculated inferred carbs in calculating predicted blood glucose levels, which may be used to issue alarms or alerts (e.g., predictive hypoglycemic or predictive hyperglycemic alarms or alerts) to the PWD. In some cases, a Recommended Correction Bolus may be negative. In some cases, devices, systems, and methods provided herein can calculate an amount of inferred carbs by multiplying the bolus by the CR when the user does not input or retrieve an EGV and/or does not have the system calculate a recommended correction bolus. By having methods, systems, and devices infer a number of carbohydrates, methods, devices, and systems provided herein can match the user's mental model without requiring the user to enter data.

### Systems for the Treatment of Type 2 Diabetes

In some cases, methods, systems, and devices provided herein can be used to treat a person with type 2 diabetes (PWT2D) and to personalize insulin therapy for the treatment of Type 2 Diabetes (T2D). For example, the system shown in FIG. 1A can be used for the treatment of a PWT2D.

Type 2 Diabetes is often treated by slowly adding treatments. Initially, a PWT2D may be advised to control their diet and to exercise in order to prevent high blood glucose levels, which could be reviewed by logging blood glucose readings taken with a BGM. If diet and exercise is insufficient to achieve glycemic control, which may be defined by an HBA1C value of less than 7% and fasting/pre-meal blood glucose readings of less than 110 mg/dL (but may be personalized based on a number of factors), then the PWT2D may begin treatment of various drugs like GLP-1 RA or SGLT-2i or DPP-4i, which are designed to lower blood glucose levels. If those drugs do not achieve appropriate glycemic control, then the PWT2D may start insulin therapy using one or two injections of LAI, which or without the use of other drugs. Systems, devices, and methods provided herein can be used to assist PWT2Ds with the creating of a data log of blood glucose readings, documenting meals, and reminders of when to take post-meal blood glucose readings even if the PWT2D is not on insulin therapy.

If the PWT2D is taking LAI but not QAI for meals, methods and systems provided herein can be used to make adjustments to the LAI injections in addition to documenting BGM data and meals and issuing reminders. When starting systems and methods provided herein, the user and/or provider of healthcare services may set the initial amounts of LAI based on the PWT2D's previous LAI therapy. If the PWT2D is starting LAI therapy for the first time, the total units of LAI per day may be set at about 0.2 U/kg, or any amount between 0.1 and 0.3 U/kg, when the PWT2D starts the system. For example, if a PWT2D has an A1C of less than 8%, a provider of healthcare may typically set the total LAI therapy at somewhere between 0.1 and 0.2 U/kg. If a PWT2D has an A1C of greater than 8%, a provider of healthcare may typically set the total LAI therapy at somewhere between 0.2 and 0.3 U/kg. For example, a PWT2D weighing 100 kg and having an A1C of 8% might have a provider of healthcare set a total daily dose of LAI at 20 Units (e.g., 10 Units at 8 AM and 10 Units at 8 PM). In some cases, mobile application 10 can include an interface for the PWT2D or their provider of healthcare to enter an initial LAI therapy, which could be updated or adjusted later (both by the algorithm provided below or manually). In some cases, LAI therapy could be initially set and/or updated by a provider of healthcare in a remote web interface that connects to the mobile application through the cloud. In some cases, methods and systems provided herein may require that a qualified healthcare professional enter or confirm the initial LAI therapy.

Methods, devices, and systems provided herein can use and adjust the LAI therapy by tracking blood glucose data and LAI injections. In some cases, the LAI can be upwardly adjusted if an average fasting blood glucose reading for a period of time (e.g., 1 day, 2 days, 3 days, 5 days, 7 days, or more) exceeds a threshold. In some cases, the amount of the adjustment can depend on how much the average fasting blood glucose value exceeds a threshold. For example, in some cases a 3 day average fasting blood glucose value of between 110 and 140 mg/dL would result in an increase of 1 unit LAI per day, a 3 day average fasting blood glucose value of between 140 and 180 mg/dL would result in an increase of LAI by 10%, and a 3 day average fasting blood glucose value of at least 180 mg/dL would result in an increase of LAI by 20%. In some cases, the increase in percentage of LAI can be linearly proportional to the 2 or 3 day average over 110 mg/dL.

In some cases, the LAI can be downwardly adjusted if any blood glucose reading is below a threshold value, which could be hypoglycemia or indicate a risk for hypoglycemia. The decrease can be proportional to the low blood glucose reading. In some cases, if a blood glucose reading is between 40 and 70 mg/dL, the LAI would be decreased by between 10-20%, and a blood glucose reading of less than 40 mg/dL would result in a decrease of between 20-40%. In some cases, methods and systems provided here would decrease LAI 10% for a reading of about 70 mg/dL, decrease it by 20% for a reading of about 40 mg/dL, and decrease it by 40% for a reading of about 30 mg/dL or less.

Typically, if LAI therapy is achieving glycemic control, the system should not produce contradictory upward and downward adjustments. Moreover, glycemic control should result in the absence of hypoglycemia, prevent fasting and pre-meal blood glucose readings of greater than 110 mg/dL, and an A1C of less than 7%. If methods and systems fail to achieve glycemic control after a sufficient period of time (e.g., 1 month, 2 months, etc.), which can be preset or set by a provider of healthcare, the system can send a message to the provider of healthcare to indicate that additional therapy might be considered, which may include drugs like GLP-1 RA or SGLT-2i or DPP-4i or the use of both LAI and QAI therapy. If additional drugs other that QAI are added to the therapy, the system may continue to adjust LAI as described above and determine if glycemic control is achieved after a sufficient period of time (e.g., 1 month, 2 months, etc.).

If a PWT2D switches from LAI therapy alone to therapy using both LAI and QAI, a provider of healthcare can either switch to QAI for only some meals or for all meals. For example, in some cases a provider of healthcare may reduce LAI by 10% or 5 units and set a prandial QAI bolus for the largest meal at that 10% value or the value of 5 units, potentially adding prandial QAI boluses for additional meals if that fails to achieve glycemic control. In some cases, a provider of healthcare may decide to reduce LAI by 50% and set prandial QAI boluses at values to equal the reduction in LAI, perhaps estimating different amounts for different meals. Regardless of the amounts of LAI and prandial QAI boluses and times set by the provider of healthcare, methods and systems provided herein can make adjustments to both LAI and QAI injections based on blood glucose readings. The LAI total units per day could be decreased or increased using the same criteria discussed above for any fasting blood glucose reading. Each prandial QAI bolus can be adjusted by increasing it if a running average blood glucose reading after that meal is above a high threshold and decreasing if a post-meal blood glucose reading is below a low threshold. For example, if a post-meal blood glucose reading 2 hours after a meal is between 70 and 40 mg/dL, the prandial QAI bolus for that meal would be reduced by between 10 and 20%, and it would be reduced by between 20 and 40% if it is below 40 mg/dL. Post-meal blood glucose readings above 140 mg/dL could, for example, result in the system increasing the prandial QAI bolus for that meal by 10% or between 1-2 units of QAI for that meal. Accordingly, the systems and methods presented herein can personalize the size of prandial QAI meal boluses, which may be due to a PWT2D's typical meal size or variations in insulin sensitivity and carbohydrate-to-insulin ratios during the day. Systems provided herein could also issue notices to users if the prandial QAI boluses are producing highly variable post-meal blood glucose readings, indicating to the PWT2D that the meal sizes should remain approximately constant.

Systems, devices, and methods provided herein can also flag unusual circumstances for the user or the provider of healthcare and suggest additional tasks. For example, in some cases, a user may have a post-dinner blood glucose reading of 130 mg/dL, but wake up with a fasting blood glucose reading of greater than 160 mg/dL, which may indicate that the PWT2D may be experience a nighttime low followed by a rebound in blood glucose levels due to a biological response (e.g., the release of glucagon from the liver), thus the system may suggest an occasional additional blood glucose reading at night. A nighttime low may indicate a need to adjust the dinner QAI bolus or the units of LAI. In some cases, methods and systems provided herein may have data regarding a next appointment with a provider of healthcare and ask the PWT2D to take additional blood glucose measurements for a few days leading up to the appointment.

### System-Based Inferences

In some cases, methods, devices or systems of the present disclosure may infer certain information by observing and/or analyzing data gathered in accordance with the present disclosure. For example, inferences may be made regarding whether or not a meal was consumed, a size of a meal consumed, whether or not a bolus of insulin was received, and/or a size of a bolus of insulin received.

In some cases, methods, devices or systems of the present disclosure may analyze historic blood glucose readings and note points when blood glucose levels rise, particularly around meal times. By observing rising blood glucose levels, an inference may be made regarding the consumption of a meal. Additionally or alternatively, using user-specific parameters (e.g., carbohydrate-to-insulin ratio (CR), insulin sensitivity factor (ISF), insulin-on-board (IOB), etc.) and/or historic data, a size of a meal may be inferred based on the amount of change in blood glucose levels and data gathered regarding meal sizes for the PWD. For example, if a known amount of insulin is repeatedly given for a PWD as a bolus for a meal and a known response is expected for an expected meal size, variations in that response may convey variations in the size of the meal.

In some cases, methods, devices or systems of the present disclosure may analyze historic blood glucose readings and note points when blood glucose levels decrease. For example, an inference may be made whether or not a user has received a bolus of insulin based on a decrease in blood glucose level based on an expected bolus associated with a meal. For example, an initial increase in blood glucose level around a meal time followed by a decrease in blood glucose level may indicate that a user did, in fact, receive a bolus for a meal. Additionally or alternatively, using user-specific parameters and/or historical data, a size of a bolus may be estimated. For example, if a meal size is known (or estimated) and a typical response is known (or expected) for a PWD, a decrease in blood glucose level may permit methods, devices or systems of the present disclosure to infer a bolus size. In some cases, methods, devices or systems of the present disclosure may use inferences to act as a security check to verify that a PWD received a bolus in association with a meal. For example, a PWD may use an insulin pen for boluses that is not in communication with other components of a system or device of the present disclosure, and such approaches may verify that a bolus was given for a meal.

In some cases, expected variations in blood glucose levels may incorporate the overlap of expected blood glucose levels due to LAI, QAI, and consumed carbohydrates. Such data may be inferred, read from one or more sensors or devices, or input by a user or PWD.

The embodiments described herein may include the use of a special-purpose or general-purpose computer including various computer hardware or software modules, as discussed in greater detail below.

Embodiments described herein may be implemented using computer-readable media for carrying or having computer-executable instructions or data structures stored thereon. Such computer-readable media may be any available media that may be accessed by a general-purpose or special-purpose computer. By way of example, and not limitation, such computer-readable media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable media.

Computer-executable instructions comprise, for example, instructions and data which cause a general-purpose computer, special-purpose computer, or special-purpose processing device (e.g., one or more processors) to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Any ranges expressed herein (including in the claims) are considered to be given their broadest possible interpretation. For example, unless explicitly mentioned otherwise, ranges are to include their end points (e.g., a range of “between X and Y” would include X and Y). Additionally, ranges described using the terms “approximately” or “about” are to be understood to be given their broadest meaning consistent with the understanding of those skilled in the art. Additionally, the term approximately includes anything within 10%, or 5%, or within manufacturing or typical tolerances.

All examples and conditional language recited herein are intended for pedagogical objects to aid the reader in understanding the disclosure and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the disclosure.

What is claimed is:

1. A remote user interface device comprising an insulin therapy management application and in wireless communication with a glucose sensor, the remote user interface device comprising:
a memory configured to store the insulin therapy management application; and
a processor communicatively coupled to the memory and configured to cause the insulin therapy management application to:
display a user interface comprising a plurality of user-selectable icons including a user-selectable meal icon and a user-selectable bolus icon, wherein the user-selectable meal icon is associated with a preset meal characteristic and the user-selectable bolus icon is associated with a bolus calculator function of the insulin therapy management application;
receive, subsequent to receiving a selection of the user-selectable meal icon, a selection of the user-selectable bolus icon, wherein the selection of the user-selectable bolus icon comprises a user-provided dosage amount;
receive, subsequent to receiving the selection of the user-selectable bolus icon, a post-meal glucose level from the glucose sensor;
adjust, based on the post-meal glucose level, the preset meal characteristic to form a personalized meal characteristic; and
update the user interface to display the user-selectable meal icon and the personalized meal characteristic.

2. The remote user interface device of claim 1, wherein the processor is further configured cause the insulin therapy management application to:
display a recommended dosage amount on the user interface, wherein the recommended dosage amount is different from the user-provided dosage amount.

3. The remote user interface device of claim 2, wherein the processor is further configured cause the insulin therapy management application to:
determine a difference between the post-meal glucose level to a threshold glucose level; and
display a notification on the user interface responsive to the difference being above a threshold amount, wherein the notification includes the recommended dosage amount.

4. The remote user interface device of claim 1, wherein the plurality of user-selectable icons further comprises a second user-selectable meal icon associated with a second preset meal characteristic.

5. The remote user interface device of claim 4, wherein the preset meal characteristic is associated with a first carbohydrate amount for a meal and the second preset meal characteristic is associated with a second carbohydrate amount for the meal.

6. The remote user interface device of claim 1, wherein the preset meal characteristic comprises a predetermined carbohydrate amount and the personalized meal characteristics comprises a personalized carbohydrate amount.

7. The remote user interface device of claim 1, wherein the processor is further configured cause the insulin therapy management application to detect a pattern of differences between historical recommended dosage amounts and historical user-provided dosage amounts, wherein adjusting the preset meal characteristic to form the personalized meal characteristic is further based on the detected pattern of differences.

8. A method for operating an insulin therapy management application in a remote user interface device in wireless communication with an insulin delivery device and a glucose sensor, the method comprising:
displaying a user interface comprising a plurality of user-selectable icons including a user-selectable meal icon and a user-selectable bolus icon, wherein the user-selectable meal icon is associated with an initial meal characteristic and the user-selectable bolus icon is associated with a bolus calculator function of the insulin therapy management application;
receiving, subsequent to receiving a selection of the user-selectable meal icon, a selection of the user-selectable bolus icon, wherein the selection of the user-selectable bolus icon comprises a user input regarding a dosage amount;
receiving, subsequent to receiving the selection of the user-selectable bolus icon, a post-meal glucose level provided by the glucose sensor;
adjusting, based on the post-meal glucose level, the initial meal characteristic to form a personalized meal characteristic; and
updating the user interface to display the user-selectable meal icon and the personalized meal characteristic.

9. The method of claim 8, further comprising:
displaying a recommended dosage amount on the user interface, wherein the recommended dosage amount is different from the user-provided dosage amount.

10. The method of claim 8, further comprising:
determining a difference between the post-meal glucose level to a threshold glucose level; and
displaying a notification on the user interface responsive to the difference being above a threshold amount, wherein the notification includes the recommended dosage amount.

11. The method of claim 8, wherein the plurality of user-selectable icons further comprises a second user-selectable meal icon associated with a second preset meal characteristic.

12. The method of claim 11, wherein the preset meal characteristic is associated with a first carbohydrate amount for a meal and the second preset meal characteristic is associated with a second carbohydrate amount for the meal.

13. The method of claim 8, wherein the preset meal characteristic comprises a predetermined carbohydrate amount and the personalized meal characteristics comprises a personalized carbohydrate amount.

14. The method of claim 8, further comprising:
detecting a pattern of differences between historical recommended dosage amounts and historical user-provided dosage amounts, wherein adjusting the preset meal characteristic to form the personalized meal characteristic is further based on the detected pattern of differences.

15. A non-transitory computer-readable device having instructions stored thereon that, when executed by an insulin therapy management application in a remote user interface device in wireless communication with an insulin delivery device and a glucose sensor, causes the remote user interface device to perform operations comprising:
displaying a user interface comprising a plurality of user-selectable icons including a user-selectable meal icon and a user-selectable bolus icon, wherein the user-selectable meal icon is associated with an initial meal characteristic and the user-selectable bolus icon is associated with a bolus calculator function of the insulin therapy management application;
receiving, subsequent to receiving a selection of the user-selectable meal icon, a selection of the user-selectable bolus icon, wherein the selection of the user-selectable bolus icon comprises a user input regarding a dosage amount;
receiving, subsequent to receiving the selection of the user-selectable bolus icon, a post-meal glucose level provided by the glucose sensor;
adjusting, based on the post-meal glucose level, the initial meal characteristic to form a personalized meal characteristic; and
updating the user interface to display the user-selectable meal icon and the personalized meal characteristic.

16. The non-transitory computer-readable device of claim 15, the operations further comprising:
displaying a recommended dosage amount on the user interface, wherein the recommended dosage amount is different from the user-provided dosage amount.

17. The non-transitory computer-readable device of claim 16, the operations further comprising:
determining a difference between the post-meal glucose level to a threshold glucose level; and
displaying a notification on the user interface responsive to the difference being above a threshold amount, wherein the notification includes the recommended dosage amount.

18. The non-transitory computer-readable device of claim 15, wherein the plurality of user-selectable icons further comprises a second user-selectable meal icon associated with a second preset meal characteristic.

19. The non-transitory computer-readable device of claim 18, wherein the preset meal characteristic is associated with a first carbohydrate amount for a meal and the second preset meal characteristic is associated with a second carbohydrate amount for the meal.

20. The non-transitory computer-readable device of claim 15, wherein the preset meal characteristic comprises a predetermined carbohydrate amount and the personalized meal characteristics comprises a personalized carbohydrate amount.
